(19) United States
(12) Patent Application Publication
(10) Pub. No.: US 2024/0002867 A1
(43) Pub. Date: Jan. 4, 2024

(54) BACILLUS SUBTILIS STRAIN, RECOMBINANT BACILLUS SUBTILIS STRAIN AND USE THEREOF

(71) Applicant: Jiangnan University, Wuxi City (CN)

(72) Inventors: Zhiming RAO, Wuxi City (CN); Jiajia YOU, Wuxi City (CN); Taowei YANG, Wuxi City (CN); Xuewei PAN, Wuxi City (CN); Xian ZHANG, Wuxi City (CN); Meijuan XU, Wuxi City (CN)

(21) Appl. No.: 17/935,466

(22) Filed: Sep. 26, 2022

(30) Foreign Application Priority Data
Jun. 30, 2022 (CN) 202210770685.2

(51) Int. Cl. C12N 15/75; C12N 9/04; C12N 9/90; C12N 9/88; C12N 9/92; C12N 1/20; C07K 14/32; C12P 25/00

(52) U.S. Cl. CPC C12N 15/75; C12N 9/0006; C12N 9/90; C12N 9/88; C12N 9/92; C12N 1/205; C07K 14/32; C12P 25/00; C12Y 101/01049; C12Y 503/01006; C12Y 401/99012; C12Y 503/01009; C12R 2001/125

(57) ABSTRACT

The present disclosure provides a Bacillus subtilis strain, a recombinant B. subtilis strain and use thereof, belongs to the technical field of microbial fermentation. B. subtilis strain RF1-6 provided by the present disclosure is a mutant strain with the highest riboflavin yield screened by gene modification and mutagenesis using a high riboflavin-producing strain RF1 as a starting strain, deposited at China Center for Type Culture Collection (CCTCC) under accession number M 2022565. Riboflavin yield can be increased by 22.8% compared with that of the high riboflavin-producing strain RF1.

### CROSS REFERENCE TO RELATED APPLICATION

This patent application claims the benefit and priority of Chinese Patent Application No. 202210770685.2, filed on Jun. 30, 2022, the disclosure of which is incorporated by reference herein in its entirety as part of the present application.

### TECHNICAL FIELD

The present disclosure belongs to the technical field of microbial fermentation, and particularly relates to a Bacillus subtilis strain, a recombinant B. subtilis strain and use thereof.

### BACKGROUND ART

Riboflavin, also known as vitamin B2, is a unique water-soluble vitamin featuring easy photolysis. It was first separated from whey in 1879 and named milk pigment. Riboflavin can be crystallized into an orange crystal, but pure riboflavin is insoluble in water, soluble in sodium chloride solution, freely soluble in dilute sodium hydroxide solution and alkaline solutions, and stable in strongly acid solutions. It is mainly synthesized by plants and microorganisms and is an important animal nutrient. It is an important nutrient for animals and needs to be acquired from the outside. Riboflavin is converted into two active substances in the animal body: flavin adenine dinucleotide (FAD) and flavin mononucleotide (FMN). As cofactors of enzymes of oxidoreductase oxidation pathway, they participate in a series of redox reactions, some of which are critical to aerobic cellular functions. If deficient, the biological oxidation of the body will be influenced, and metabolic disorders will occur. Riboflavin deficiency mostly manifests as inflammations in the mouth, eyes, and exterior genitalia.

Riboflavin is mainly used in industries of medicine, food additives, and feed processing, as well as clinical cancer treatment and prophylaxis. At present, riboflavin is industrially produced by widely using the microbiological fermentation inside and outside of China. Microorganisms capable of synthesizing riboflavin include bacteria, fungi, and molds. B. subtilis and Eremothecium ashbyii are mainly used as production strains in industrial production. It was reported in the existing literature that the maximum yield of riboflavin synthesized by B. subtilis was 15.7 g/L (Wang, Z., Chen, T., Ma, X., Shen, Z. and Zhao, X. Enhancement of riboflavin production with Bacillus subtilis by expression and site-directed mutagenesis of zwf and gnd gene from Corynebacterium glutamicum[J]. Bioresource Technology, 2011, 102(4): 3934-3940), but this yield still fails to meet the needs of industrial production.

### SUMMARY

In view of this, an objective of the present disclosure is to provide a B. subtilis strain, a recombinant B. subtilis strain and use thereof, and the B. subtilis strain and the recombinant B. subtilis strain provided by the present disclosure obtain high riboflavin yield.

The present disclosure provides a B. subtilis RF1-6 strain, deposited at China Center for Type Culture Collection (CCTCC) under accession number M 2022565.

The present disclosure further provides a recombinant B. subtilis strain. The recombinant B. subtilis strain uses the B. subtilis RF1-6 strain according to the foregoing solution as an original strain, and includes a recombinant plasmid; a zwf gene, a ywlf gene, and a ribBA gene are inserted into the recombinant plasmid; an original plasmid of the recombinant plasmid is preferably pMA5-sat; the zwf gene, the ywlf gene, and the ribBA gene are preferably ligated to the original plasmid in a homologous recombination manner.

Preferably, the zwf gene, the ywlf gene, and the ribBA gene are inserted between EcoRI and KpnI restriction sites of the recombinant plasmid in sequence.

Preferably, a strong promoter P₄₃ is further inserted into the recombinant plasmid.

Preferably, the strong promoter P₄₃ is inserted at the EcoRI restriction site in an upstream region of the zwf gene.

Preferably, a pgi gene is knocked down from the recombinant B. subtilis strain.

Preferably, a purR gene is knocked down from the recombinant B. subtilis strain.

Preferably, the recombinant B. subtilis strain includes a B. subtilis RF1-6ZYRS strain, deposited at CCTCC under accession number M 2022566.

The present disclosure further provides a microbial inoculant, including the B. subtilis strain according to the foregoing solution or the recombinant B. subtilis strain according to the foregoing solutions.

The present disclosure further provides use of the B. subtilis RF1-6 strain according to the foregoing solution, the recombinant B. subtilis strain, or the microbial inoculant in synthesis of riboflavin.

The present disclosure provides a B. subtilis RF1-6 strain. The B. subtilis RF1-6 strain provided by the present disclosure is a mutant strain with the highest riboflavin yield screened by gene modification and mutagenesis using a high riboflavin-producing strain RF1 as a starting strain, deposited at CCTCC under accession number M 2022565. Riboflavin yield can be increased by 22.8% compared with that of the high riboflavin-producing strain RF1.

The present disclosure further provides an engineering B. subtilis RF1-6ZYRS strain constructed based on the B. subtilis RF1-6 strain. The maximum riboflavin yield of the B. subtilis RF1-6ZYRS strain reaches 25.2 g/L, which is increased by 69.58% compared with the final yield of the B. subtilis RF1-6 strain.

Deposit of Biological Material

Bacillus subtilis RF1-6 was deposited at CCTCC, No. 299, Bayi Road, Wuchang District, Wuhan City, Hubei Province on May 9, 2022 under accession number M 2022565.

Bacillus subtilis RF1-6ZYRS was deposited at CCTCC, No. 299, Bayi Road, Wuchang District, Wuhan City, Hubei Province on May 9, 2022 under accession number M 2022566.

### BRIEF DESCRIPTION OF THE DRAWINGS

FIG. 1 illustrates a mutagenized plasmid provided by the present disclosure;

FIG. 2 illustrates the working principle of a report system;

FIG. 3 illustrates shake-flask rescreening results;

FIG. 4 illustrates the fermentation result of mutant strain RF1-6 in a fermentor;

FIG. 5 illustrates the fermentation result of microbial inoculant RF1-6ZYPS in a 5 L fermentor.

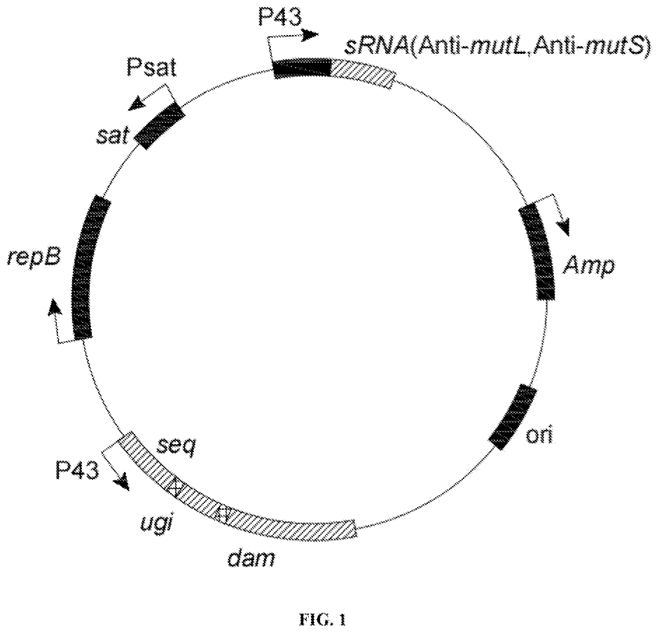

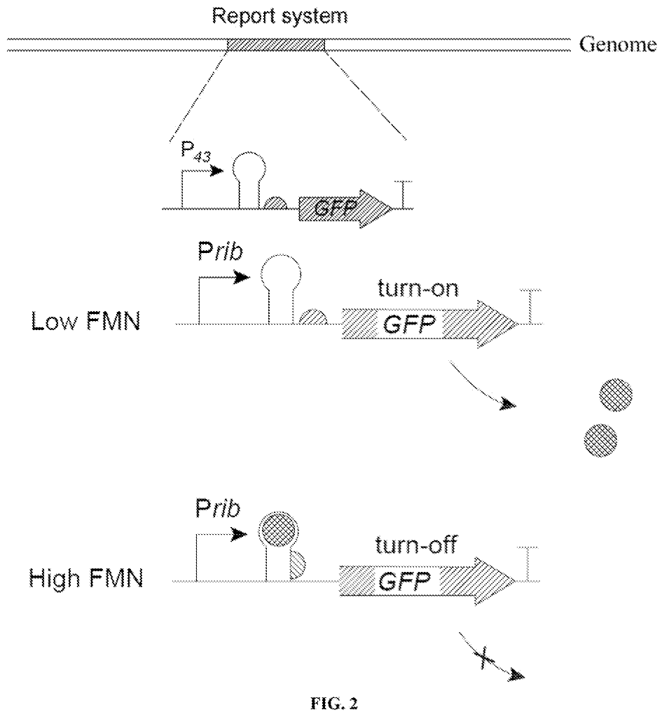

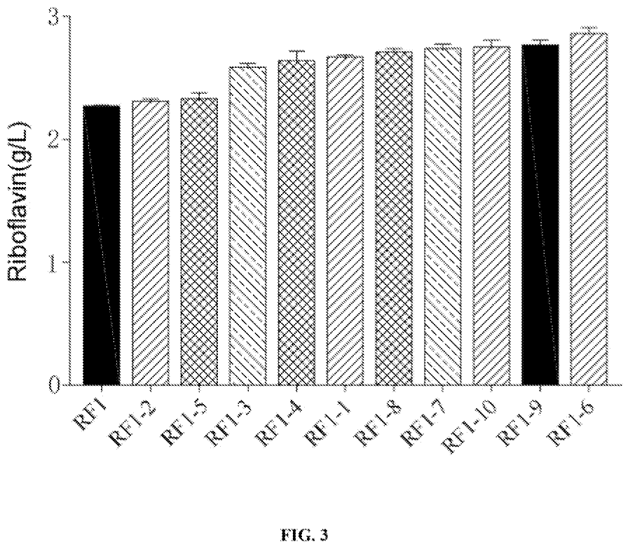

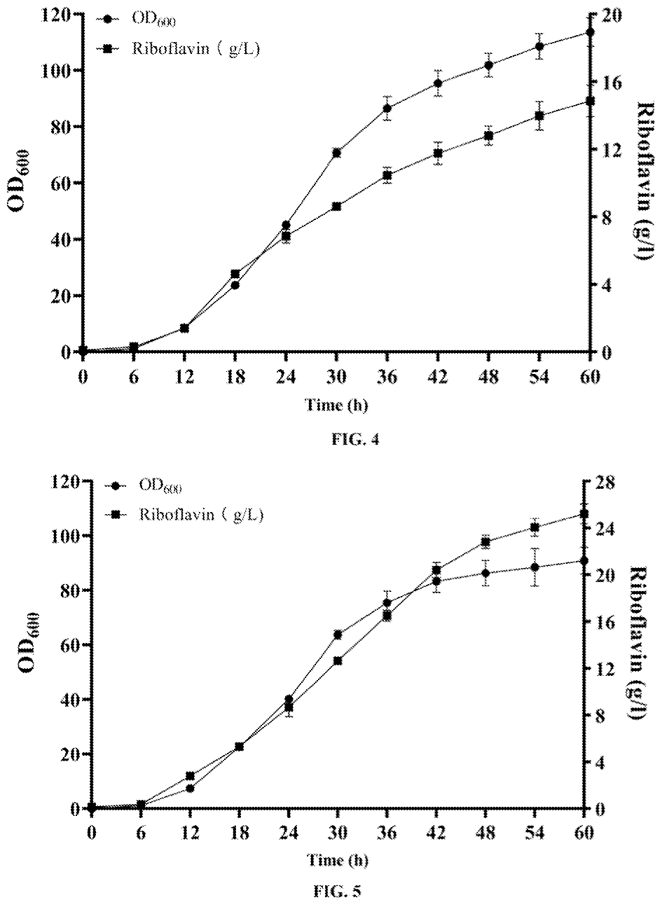

### DETAILED DESCRIPTION OF THE EMBODIMENTS

The present disclosure provides a B. subtilis RF1-6 strain, deposited at CCTCC under accession number M 2022565.

In the present disclosure, the B. subtilis RF1-6 strain is a mutant strain with a highest riboflavin yield screened by gene modification and mutagenesis using a high riboflavin-producing strain RF1 as a starting strain. Riboflavin yield may be increased by 22.8% compared with that of the high riboflavin-producing strain RF1.

The present disclosure further provides a recombinant B. subtilis strain. The recombinant B. subtilis strain uses the B. subtilis RF1-6 strain according to the foregoing solution as an original strain, and includes a recombinant plasmid; a zwf gene, a ywlf gene, and a ribBA gene are inserted into the recombinant plasmid.

Through metabolic engineering of a high riboflavin-producing strain RF1-6, the present disclosure increases a metabolic flux of the riboflavin and thus the riboflavin yield.

In the present disclosure, the zwf gene, the ywlf gene, and the ribBA gene are preferably inserted between EcoRI and KpnI restriction sites of the recombinant plasmid in sequence; a strong promoter P₄₃ is further preferably inserted into the recombinant plasmid; the strong promoter P₄₃ is preferably inserted at the EcoRI restriction site in an upstream region of the zwf gene. An original plasmid of the recombinant plasmid is preferably pMA5-sat; the zwf gene, the ywlf gene, and the ribBA gene are preferably ligated to the original plasmid in a homologous recombination manner.

In the present disclosure, the zwf encodes glucose-6-phosphatase dehydrogenase, the ribBA encodes a bifunctional cyclohydrolase II/3,4-dihydroxy-2-butanone 4-phosphate synthase, and the ywlf encodes ribose-5-phosphate isomerase B. These three genes jointly promote the metabolic flux of riboflavin synthesis and increase the riboflavin yield.

In the present disclosure, the strong promoter P₄₃ is used to control the expression of the zwf gene, the ywlf gene, and the ribBA gene.

In the present disclosure, a pgi gene is preferably knocked down from the recombinant B. subtilis strain. With an sRNA knockdown technique, an expression level of the pgi gene is downregulated to make the metabolic flux flow to a phosphopentose pathway. In the present disclosure, a purR gene is preferably knocked down from the recombinant B. subtilis strain. Intracellular feedback inhibition may be relieved by purR knockout, resulting in intracellular biosynthesis of more precursor GTPs.

In the present disclosure, the recombinant B. subtilis strain preferably includes a B. subtilis RF1-6ZYRS strain, deposited at CCTCC under accession number M 2022566.

In the present disclosure, a maximum riboflavin yield of the B. subtilis RF1-6ZYRS strain reaches 25.2 g/L, which is increased by 69.58% compared with a final yield of the B. subtilis RF1-6 strain.

The present disclosure further provides a microbial inoculant, including the B. subtilis strain according to the foregoing solution or the recombinant B. subtilis strain according to the foregoing solutions.

The present disclosure further provides use of the B. subtilis RF1-6 strain according to the foregoing solution, the recombinant B. subtilis strain, or the microbial inoculant in synthesis of riboflavin.

The technical solutions of the present disclosure will be described below clearly and completely in conjunction with the examples of the present disclosure.

Culture media used in the following examples are shown below:

LB agar includes: 10 g/L peptone, 5 g/L yeast extract paste, 10 g/L NaCl, and 0.2 g/L agar powder.

LB broth includes: 10 g/L peptone, 5 g/L yeast extract paste, and 10 g/L NaCl.

Shake-flask fermentation medium includes: 20 g/L glucose, 20 g/L yeast powder, 4 g/L ammonium citrate, 1 g/L K₂HPO₄, 1 g/L KH₂PO₄, 2 g/L MgSO₄·7H₂O, 0.04 g/L MnCl₂, 0.06 g/L CaCl₂), and 2 g/L CuSO4, at pH 6.8.

Inoculum medium includes: 40 g/L glucose, 5 g/L yeast extract paste, 10 g/L peptone, 10 g/L NaCl, and 10 μg/mL chloramphenicol.

Fermentation medium for fed-batch culture includes: 20 g/L glucose, 20 g/L yeast powder, 6 g/L (NH₄)₂HPO₄, 5 g/L K₂HPO₄, 1.5 g/L MgSO₄·7H₂O, 0.03 g/L ZnSO₄·7H₂O, 0.05 g/L MnCl₂, and 0.02 g/L FeSO₄·7H₂O.

Fed-batch medium includes: 600 g/L glucose, 10 g/L yeast powder, 6 g/L (NH₄)₂HPO₄, 5 g/L K₂HPO₄, and 0.5 g/L MgSO₄·7H₂O.

All of the above culture media use water as a solvent.

Detection methods used in the following examples are as follows:

Cell growth is monitored at OD₆₀₀ using a spectrophotometer.

A prepared fermentation broth is diluted with 0.01 M NaOH and centrifuged at 12,000 rpm for 2 min, and a supernatant is collected to determine a riboflavin concentration. The supernatant is transferred into a new EP tube and diluted to a suitable concentration range (0.3 to 0.8), and an absorbance value (OD) is measured at 444 nm using a spectrophotometer. The riboflavin concentration is calculated according to a standard curve of riboflavin concentration. According to the riboflavin standard curve, the calculation formula is: OD₄₄₄*dilution multiple*30/1,000.

Glucose concentrations are determined by Glucose analysis (Model-SBA40, Shandong, China).

### Example 1 Construction of a Mutagenized Plasmid

1. Construction of a mutagenized plasmid: The dam and seq genes were amplified on the plasmid MP6. The ugi gene was derived from Escherichia coli genome. The PCR product was separated by agarose gel electrophoresis, and the target PCR product was recovered by gel extraction. Three fragments were fused by overlap extension PCR. First, upstream and downstream fragments were mixed in a volume ratio of 1:1, and isometric PCR enzyme was added for fusion PCR. The reaction conditions were as follows: initial denaturation at 98° C. for 3 min; 34 cycles of denaturation at 98° C. for 10 s, annealing at 58° C. for 15 s, and extension at 72° C. for 1 min. The PCR product was recovered, and the fusion fragment was ligated to HindIII and BamHI sites of the pBT2 plasmid using a Gibson Assembly kit; gene expression was controlled by strong promoter P₄₃ to construct a pBT2-M3 plasmid. The pBT2 plasmid was a temperature-sensitive plasmid, which was lost at 42° C., without impact on the genetic stability of the mutant strain. The mutL gene was derived from E. coli. The gene was amplified and ligated to the pET28a plasmid to construct a pET28a-mutL plasmid according to the above method. The pET28a-mutL plasmid was amplified with a reverse amplification primer. There was a mutation site on the primer. Mutation was introduced on the mutL gene to construct a pET28a-mutL (E32K) plasmid with plasmid mutation. The PCR fragment of the mutL gene was amplified with a well-constructed mutated mutL gene, and the mutated mutL gene was ligated to the pBT2-M3 plasmid to construct a mutagenized plasmid pBT2-M4 in the above Gibson assembly manner.

2. Construction of a reporter plasmid: The FMNswitch sequence at 5′-terminal upstream the rib operon was cloned from the B. subtilis 168 genome, while the reporter gene gn, was amplified; the FMN riboswitch was fused with gn, to form a fused FMNswitch-gfp fragment according to the above overlap extension PCR. The upstream homologous arm (1,000 bp) and downstream homologous arm (1,000 bp) of the amyE gene and the Marker fragment (containing a bleomycin resistance gene and a lox66-lox71 recombination site) were amplified, the PCR product was separated by agarose gel electrophoresis, and the target PCR product was recovered by gel extraction. According to the above overlap extension PCR method, the fused PCR fragment was integrated into the genome of the high riboflavin-producing strain RF1.

3. Construction of a mutant library: The mutagenized plasmid pBT2-M4 was introduced into the high riboflavin-producing strain RF1 containing a reporter plasmid; cells were cultured until the logarithmic phase (OD₆₀₀=0.6), and the cells at logarithmic phase were mutagenized by atmospheric room temperature plasma (ARTP); the treated mutant library was cultured in inorganic salt medium for 12 h and sampled, and cells were collected and washed with phosphate-buffered saline (PBS) thrice; the washed cells were reselected with PBS, diluted to a suitable bacterial concentration, and sorted by flow cytometry; a flora with a lower fluorescence intensity than the control strain (ARTP untreated) was selected and subcultured.

4. The separated mutant was spread on a resistant plate and cultured at 37° C. for 24 h; using a high-throughput colony picking system, a colony with weak fluorescence intensity on the plate was picked into a 96-well plate with inorganic salt medium; after shake culture for 24 h, the absorbance value at OD₄₄₄ was measured, at which riboflavin has a maximum absorption peak that can indirectly reflect the riboflavin concentration. One thousand cells were picked for rescreening identification each round. The riboflavin synthesis ability (OD₄₄₄/OD₆₀₀) was calculated, and a mutant strain with the highest yield was picked.

5. Shake-flask rescreening: the screened mutant strain was inoculated into a 250 mL shake flask with 50 mL of fermentation medium and cultured at 200 r/min and 41° C. for 48 h; the riboflavin concentration was determined, and a mutant strain with the highest yield was rescreened.

6. The screened mutant strain was streaked on the plate and purified; a purified colony was inoculated on LBG medium and cultured at 42° C., the mutagenized plasmid was discarded, and shake-flask culture was conducted to determine its final yield. After rescreening, 10 mutant strains with the highest yield were subjected to shake-flask culture. Results showed that the riboflavin yield of the mutant strain with the highest yield was increased by 22.8%, and the mutant strain was named RF1-6 and deposited at CCTCC under accession number M 2022565.

The riboflavin yield of the mutant strain was identified by a 5 L fermentor. The riboflavin yield of the high riboflavin-producing strain RF1-6 screened by the shake-flask experiment was determined by the 5 L fermentor. Specific steps were as follows:
(1) The B. subtilis RF1-6 strain cultured on 10 mL of LB broth for 24 h was inoculated in 100 mL of inoculum medium with a 3% v/v inoculum size, and cultured at 180 rpm and 41° C. for 16 h to prepare an inoculum (OD₆₀₀=21.2); and
(2) 100 mL of the prepared inoculum was all inoculated into a 5 L fermentor with 1,900 mL of fermentation medium to conduct fed-batch culture.

By controlling the flux of the fed-batch medium, the concentration of the remaining glucose in the fermentation broth was maintained at not less than 5 g/L. During fermentation, the pH value of the fermentation broth was 6.8, and 1 M H₂SO₄ and 50% ammonia water were added. Before batch feeding, the rotational speed was maintained at 400 rpm and then increased to 800 rpm until the end of the fermentation, while the temperature was always held at 41° C. After fed-batch culture for 60 h, the riboflavin yield was determined, and the riboflavin concentration reached 14.86 g/L. Compared with the riboflavin concentration (9.8 g/L) of the high riboflavin-producing strain RF1 (starting strain), the yield was increased by 48.9%. Therefore, the mutagenesis system and the high-throughput screening system can effectively improve the performance of industrial strain and increase metabolite synthesis.

7. To further increase the riboflavin yield, metabolic engineering was conducted on the high-producing strain by conventional genetic manipulation, increasing the metabolic flux of riboflavin and the riboflavin yield. First, an overexpression plasmid was constructed. The zwf,ywlf, and ribBA genes were ligated to the pMA5-sat plasmid in a homologous recombination manner, and gene expression was controlled by strong promoter P₄₃ to construct a microbial inoculant RF1-6ZYP; subsequently, the expression level of the pgi gene was knocked down by the sRNA knockdown technique to make the metabolic flux flow to a phosphopentose pathway. To increase the synthesis of precursor GTP, intracellular feedback inhibition was relieved by purR knockout, resulting in intracellular biosynthesis of more precursor GTPs. The resulting microbial inoculant was named RF1-6ZYRS, deposited at CCTCC under accession number M 2022566. The microbial inoculant obtained by a series of metabolic engineering substantially increased the riboflavin yield at a 5 L fermentor level, where its maximum yield reached 25.2 g/L, and its final yield was increased by 69.58%.

Although the above example has described the present disclosure in detail, it is only a part of, not all of, the examples of the present disclosure. Other examples may also be obtained by persons based on the example without creative efforts, and all of these examples shall fall within the protection scope of the present disclosure.

1. A recombinant Bacillus subtilis strain, using Bacillus subtilis RF1-6 strain as an original strain, and comprising a recombinant plasmid, wherein zwf gene, a ywlf gene, and a ribBA gene are inserted into the recombinant plasmid;
an original plasmid of the recombinant plasmid is preferably pMA5-sat;
the zwf gene, the ywlf gene, and the ribBA gene are preferably ligated to the original plasmid in a homologous recombination manner; and
the Bacillus subtilis RF1-6 strain is deposited at China Center for Type Culture Collection (CCTCC) under accession number M 2022565.

2. The recombinant Bacillus subtilis strain according to claim 1, wherein the zwf gene, the ywlf gene, and the ribBA gene are inserted between EcoRI and KpnI restriction sites of the recombinant plasmid in sequence.

3. The recombinant Bacillus subtilis strain according to claim 2, wherein a strong promoter P₄₃ is further inserted into the recombinant plasmid.

4. The recombinant Bacillus subtilis strain according to claim 3, wherein the strong promoter P₄₃ is inserted at the EcoRI restriction site in an upstream region of the zwf gene.

5. The recombinant Bacillus subtilis strain according to claim 2, wherein a pgi gene is knocked down from the recombinant Bacillus subtilis strain.

6. The recombinant Bacillus subtilis strain according to claim 3, wherein a pgi gene is knocked down from the recombinant Bacillus subtilis strain.

7. The recombinant Bacillus subtilis strain according to claim 4, wherein a pgi gene is knocked down from the recombinant Bacillus subtilis strain.

8. The recombinant Bacillus subtilis strain according to claim 2, wherein a purR gene is knocked down from the recombinant Bacillus subtilis strain.

9. The recombinant Bacillus' subtilis strain according to claim 3, wherein a purR gene is knocked down from the recombinant Bacillus subtilis strain.

10. The recombinant Bacillus subtilis strain according to claim 4, wherein a purR gene is knocked down from the recombinant Bacillus subtilis strain.

11. The recombinant Bacillus subtilis strain according to claim 4, wherein the recombinant Bacillus subtilis strain comprises a Bacillus subtilis RF-6ZYRS strain, deposited at CCTCC under accession number M 2022566.

12. A microbial inoculant, comprising the Bacillus subtilis strain according to claim 1.

13. A microbial inoculant, comprising the recombinant Bacillus subtilis strain according to claim 2.

14. The microbial inoculant according to claim 13, wherein a strong promoter P₄₃ is further inserted into the recombinant plasmid.

15. The microbial inoculant according to claim 14, wherein the strong promoter P₄₃ is inserted at the EcoRI restriction site in an upstream region of the zwf gene.

16. The microbial inoculant according to claim 13, wherein a pgi gene is knocked down from the recombinant Bacillus subtilis strain.

17. The microbial inoculant according to claim 14, wherein a pgi gene is knocked down from the recombinant Bacillus subtilis strain.

18. The microbial inoculant according to claim 15, wherein a pgi gene is knocked down from the recombinant Bacillus subtilis strain.

19. The microbial inoculant according to claim 13, wherein a purR gene is knocked down from the recombinant Bacillus subtilis strain.

20. The microbial inoculant according to claim 14, wherein a purR gene is knocked down from the recombinant Bacillus subtilis strain.
